(19) United States
(12) Patent Application Publication
(10) Pub. No.: US 2011/0257522 A1
(43) Pub. Date: Oct. 20, 2011

(54) Injection of Fluid

(75) Inventors: Nicolay Berard-Andersen, Oslo (NO); Gjermund Fjeld Olsen, Oslo (NO); Knut Brabrand, Oslo (NO)

(73) Assignee: NEORAD AS, Olso (NO)

(21) Appl. No.: 13/125,057

(22) PCT Filed: Nov. 6, 2009
(86) PCT No.: PCT/GB2009/002629

§ 371 Date: Jul. 8, 2011

Related U.S. Application Data
(60) Provisional application No. 61/112,021, filed on Nov. 6, 2008.

(30) Foreign Application Priority Data
Nov. 7, 2008 (GB) 0820471.1
Jan. 13, 2009 (GB) 0900497.9

(51) Int. Cl. A61B 8/12

(52) U.S. Cl. 600/438

(57) ABSTRACT

A system for monitoring the intended injection of a fluid into a fluid conduit in the body, wherein the fluid to be injected may include a first fluid, the system: introducing a pulse into the fluid to be injected and monitoring the flow characteristics of fluid in the fluid conduit in the body at a location downstream of the point of injection.

The invention relates to a method of monitoring the injection of a fluid in a fluid conduit in the human or animal body and to the supply of fluid for such injection.

When fluid is injected into a blood vessel there is a risk of extravasation occurring. Extravasation is the accidental infusion of a fluid such as a contrast medium or drug into tissue surrounding a blood vessel, rather than into the blood vessel itself. Extravasation can result from various situations. A common cause is operator error in placement of the needle, or movement of the needle caused by patient movement, which results in the needle effectively missing the blood vessel. In this case the injected fluid is injected into the surrounding tissue instead of into the blood vessel. Extravasation can also occur through the inability of the blood vessel to tolerate the rate of injection of the fluid resulting in leakage through the walls of the blood vessel into the surrounding tissue. For some injected fluids, such as cytotoxins, it is thought that the toxin itself may cause deterioration of the blood vessel wall and hence cause a leakage. There is a higher risk of extravasation with older patients or patients undergoing chemotherapy treatment. If an extravasation occurs then the injection and the associated procedure often has to be aborted and repeated at a later stage. In addition, some drugs can cause cell or tissue damage.

Clearly, it is beneficial to be able to detect extravasation in order to prevent pain or injury to the patient and to allow the injection procedure to be stopped if required. It is also beneficial to be able to monitor an injection to detect other problems that may occur during the course of the infusion of fluid, for example an equipment failure.

The applicant's earlier application, WO 2004/052431, discloses the use of an ultrasound Doppler technique to monitor an injection and provide a signal to indicate the possibility of extravasation. The method described is based on the fact that during the injection process the flow velocity of fluid in the blood vessel increases. Normally the flow velocity in a peripheral vein is very low, but during contrast injection using a power injector the flow velocity increases significantly. A direct monitoring of the increased venous flow velocity induced by the infusion can be performed. A lack of a velocity increase when injection commences indicates that extravasation could be occurring and thus gives the operator an early warning, prior to the point when extravasation would be detectable from a visual inspection or by palpation. The device disclosed can send an automated signal to the power injector to halt the infusion if no velocity increase is detected, thereby limiting the risk of tissue damage.

However, although this prior art system provided a major advance, there are limits on how effectively it can monitor the injection of substances at low flow rates. It has been found that extravasation can occur during injections of cytotoxins, which are used during cancer treatments. Extravasation of a vesicant cytotoxic agent is particularly serious for the patient, and will most likely result in necrosis. However, due to the low volume of cytotoxin that is typically required, and the low flow rate of the injection it is hard to detect an extravasation based on a change in flow velocity in the blood vessel, as the volume flow rate of injected fluid is small relative to the volume flow rate of the blood. Typically, flow rates of 0.1 ml/hour to 1500 ml/hour are used for cytotoxins. These flow rates are considerably lower than the flow rates of contrast medium injections. The existing system has difficulties in detecting the effect that very low flow rates have on the flow velocity in a blood vessel. Flow rates below about 360 ml/hour (about 0.1 ml/second) may be completely invisible with existing systems. There is hence a need for a way to determine if the injection of a substance at low flow rates, such as a cytotoxin, is occurring correctly.

Viewed from a first aspect, the present invention provides a method of monitoring the intended injection of a fluid into a fluid conduit in the body, wherein the fluid to be injected comprises a first fluid, the method comprising: introducing a pulse into the fluid to be injected and monitoring the flow characteristics of fluid in the fluid conduit in the body at a location downstream of the point of injection.

With the system of WO 2004/052431, where the flow rate of the injected fluid is too low to be accurately detected relative to the normal flow velocity in the blood vessel, then it is not possible to determine a possible extravasation or other problem by a change in the measured flow velocity or other flow characteristic within the blood vessel. In the present - invention, a pulse is provided to temporarily change the flow rate and/or other flow characteristics of the injected fluid. Fluid injected with a pulse applied to it has a more readily detectable influence on the flow characteristics of fluid in the fluid conduit than a conventional injection of the fluid at a fixed flow rate, with no pulse introduced into the fluid. This influence may take the form of an increase and/or decrease in the flow velocity in the fluid conduit. There may also or alternatively be an increase in the turbulence in the fluid flow. If the injection is proceeding satisfactorily then this can be detected by monitoring the fluid flow characteristics in the fluid conduit and determining if the pulse results in an expected effect on the monitored flow characteristic. If the pulse has no effect or the effect is smaller than expected, then this indicates a potential problem. The use of a pulse hence provides a positive indication of a successful injection of fluid, as the absence of an expected change in the measured flow characteristics indicates a problem. This advantage is obtained even when the flow rate of the first fluid is high enough to be detected using the prior art method.

The pulse may be introduced by any suitable means.

In one embodiment, the pulse, or more preferably a series of pulses, is introduced by applying a vibration to the tubing that supplies the fluid, or more preferably by applying a vibration to the fluid within the tubing. An vibration source such as oscillating device or motor may be used. Preferably, the vibration is at a predetermined frequency, which may be between 10 Hz and 200 Hz. If the predetermined frequency is detected in the blood vessel down stream of the injection when monitoring the injection then there are no problems with the injection. If the vibrating frequency is not detected in the fluid conduit then this indicates a potential problem somewhere along the fluid path or a potential extravasation.

In embodiments where the vibration is applied via the tubing, the tubing may be compressed by a vibration apparatus, or the tubing may be vibrated from side to side or up and down. However, it has been found that it can be difficult to isolate a vibration of the tubing from the body, to ensure that any vibration at the fluid conduit in the body is a result of passage of the first fluid into the body rather than as a result of vibrations transmitted via the tubing and catheter to the body tissue.

Hence, it is preferred that the vibration is introduced into the fluid whilst isolating the tubing and/or catheter connected to the body tissue from the source of the vibration. A vibration may be introduced in the fluid in the tubing by vibrating a syringe piston/plunger while the syringe is fluidly connected to the fluid that is being conveyed to the body. The syringe may contain the infusion fluid or may be connected directly to the fluid path using a three way stopcock/valve or the like. In one preferred arrangement, a vibration is applied to the syringe that provides the injection apparatus for the injected fluid.

Preferably, the step of introducing a pulse comprises applying a vibration to the fluid to be injected and the method includes providing damping means to reduce or eliminate the vibration transmitted to the body via tubing conveying the fluid to the body. The damping means may be provided in the form of a resilient and/or vibration absorbing material placed between the tubing and the vibration source. Various possible arrangements are discussed below. The method may include matching the frequency of vibration to the properties of the resilient and/or vibration absorbing material, such that the material is adapted to reduces or eliminates vibrations at this particular frequency. Silicone rubber material, for example a silicone rubber foam, may be used as the resilient and/or vibration absorbing material.

In an alternative embodiment, the pulse in the fluid to be injected may be provided by a second fluid injected along with the first fluid. The pulse of the second fluid can provide a temporary increase in the flow velocity of the fluid to be injected.

The method includes the step of monitoring the flow characteristics in the fluid conduit at a location downstream of the point of injection This monitoring is done by non-invasive methods. The step of monitoring may include measuring the flow velocity in the fluid conduit at a location downstream of the point of injection. This provides an effective way to measure the effect of the temporary increase and/or decrease in flow rate of the injected fluid, as there will be a corresponding change in the flow rate in the fluid conduit. For example, a vibration applied at a certain frequency will results in a corresponding fluctuation in a flow velocity measurement. Any suitable technique for measuring flow velocity may be used. Alternatively or in addition, the method may include monitoring other fluid flow characteristics, such as the turbulence generated as the injected fluid mixes with the fluid flowing through the fluid conduit. The level of turbulence may be monitored by ultrasound measurements, because the addition of the pulse can increase the number of ultrasound reflection planes (typically areas of transition between fluid types), which produces a different signal response from an ultrasound probe. In a particularly preferred embodiment, the method includes monitoring the fluid flow in the fluid conduit using a Doppler ultrasound measurement. Doppler ultrasound techniques can measure a flow velocity change and will also provide an indication of an increase in reflection planes, as this will affect the signal response. Thus, with the use of a Doppler ultrasound measurement, a velocity change and/or an increase in reflection planes can be detected. If a pulse is applied by means of a vibration, then the frequencies within the Doppler ultrasound measurement can be used to determine if the vibration has been passed into the fluid conduit in the body via the injected fluid.

In further alternative embodiments, the change in flow conditions following the introduction of the pulse may be detected by other means. For example, the pulse will produce a change in the sound of the flow in the fluid conduit, and the method may hence include monitoring the sound of the flow. A microphone and/or a stethoscope may be used.

The invention is particularly of benefit in the monitoring of an injection into a blood vessel to identify the onset of extravasation, due to the serious injury to the patient that can result. However, the invention may also be used to identify other problems with the injection process, such as a blockage or kink in the tubing conveying fluid to the patient.

The first fluid will typically be a fluid that is being injected for treatment of a patient or a fluid required as part of an imaging or diagnostic procedure. For example, the first fluid may be a cytotoxic agent, a chemotherapy drug, an analgesic, a radiopharmaceutica or different contrast media, such as XR, MR, and/or optical contrast media.

Where a pulse of a second fluid is used to supply the required temporary change in flow velocity, the second fluid is preferably a fluid that can be introduced along with the first fluid without any adverse effect on the patient and/or on the treatment, imaging or diagnostic procedure. For example, the second fluid may be a saline solution, in particular a normal saline solution of 0.9% weight/volume of NaCl, especially where the fluid conduit is a blood vessel. Other compatible injection solutions may also be used, such as a 5% glucose solution.

It is to be noted that the method relates to monitoring of an ongoing injection, and hence does not include the surgical step of insertion of a needle or catheter or the like into the body. Monitoring flow conditions using ultrasound techniques or the sound of the flow is non-invasive. When an injection of the first fluid is ongoing, the flow in the fluid conduit may be monitored. The pulse is introduced into the fluid to be injected to facilitate monitoring of the injection process.

Where a second fluid is used, the pulse of the second fluid is preferably at a higher flow rate than the flow rate of the first fluid. Any higher flow rate of the second fluid compared to the flow rate of the first fluid can be used to produce an improvement in measurability, and hence an improvement in the efficacy of the monitoring process. The pulse of the second fluid may have a flow rate of at least twice the flow rate of the first fluid, preferably at least five or ten times the flow rate. In a preferred embodiment, the flow rate of the pulse of the second fluid is at least 0.1 ml/s, preferably at least 0.2 ml/s. Typically, values from 0.1 to 1 ml/s might be used. The pulse may have a duration of at least 0.1 seconds, for example a duration between 0.1 and 1 seconds. Preferably the pulse has a duration of about 0.5 seconds, or more.

The pulse may be introduced manually by the operator. For example, a syringe can be used to provide a pulse of a second fluid. Preferably however, the method comprises introducing the pulse automatically, for example by using an infusion pump apparatus or the like and/or a vibration apparatus as discussed above. This ensures that the characteristics of the pulse(s) can be accurately controlled, thus allowing an expected change in the measured flow characteristics to be determined.

In a preferred embodiment the pulse is repeated during the course of the intended injection of the first fluid. This can be more readily achieved using an automated system, which may for example be programmed to repeat an injection of a pulse of fluid of set flow rate and duration at a predetermined interval. For example, a pulse at 0.2 ml/s lasting 0.5 seconds may be repeated every 2 seconds. By repeating the pulse, the injection of the first fluid can be monitored over a period of time, so that problems which occur part way through the injection can be detected. Alternatively, a vibration at a predetermined frequency can be maintained throughout the course of an injection of fluid.

The method may include providing feed back to the operator and/or feed back to an infusion pump apparatus that supplies the first fluid and/or the second fluid. This enables a quicker response to potential problems in the injection process, and also the use of an automated response from the infusion pump apparatus if required. The feed back may be based on the sound of the flow in the fluid conduit, and hence may simply be a reproduction of this sound with appropriate amplification. The feed back may include an indication of the measured fluid flow velocity or other flow characteristics, for example a visual or audible indication based on an ultrasound measurement, so that an operator can more easily check for the expected change in flow characteristics after the pulse is introduced such as the predetermined frequency of the vibration, or a change resulting from the pulse of second fluid. The method may include automatic checking for this change, for example by use of a controller, such as a computer device. The controller may compare the measured sound or flow velocity to an expected sound or flow velocity profile calculated based on the predetermined frequency or the size of the applied pulse of second fluid. The feed back may include providing an alarm if an expected change in the measured fluid flow characteristics is not detected or if the size of the change differs from the expected change. The method may include sending a signal to an infusion pump apparatus to automatically stop the injection when a problem is detected.

Where a second fluid is used, the first and second fluid should preferably be injected at the same location so as to ensure that the passage of the second fluid into and along the fluid conduit will be affected by any extravasations that affect the first fluid. It is possible to do this using two separate catheters, but it is preferable that the first and second fluid are introduced into the fluid conduit through a single catheter. This means that any problem that prevents effective injection of the first fluid will also prevent effective injection of the second fluid. The method may include the use of an automated injection apparatus to convey the first fluid and the second fluid to the catheter. Thus, first and second pump devices may be used for the first and second fluids, with the two pump outputs being connected to a single catheter for supplying both fluids to the fluid conduit in the body. A racked infusion pump apparatus may be used, with two or more pump devices, such as automatically controllable syringes, in a rack.

The first fluid and the pulse of the second fluid may be injected consecutively, with, for example, a period of injection of the first fluid, followed by a pulse of the second fluid, followed by recommencement of injection of the first fluid. Preferably however, the pulse of the second fluid is introduced whilst the injection of the first fluid is ongoing.

In an alternative to the first aspect above, the method of the invention may comprise a method of supplying a fluid for injection into a fluid conduit in the body, wherein the fluid to be injected comprises a first fluid, the method comprising: introducing a pulse into the fluid to be injected in order to temporarily change the flow rate of the injected fluid and conveying the fluid the fluid incorporating the pulse into the fluid conduit of the body.

Viewed from a second aspect, the present invention provides an apparatus for supplying fluid for injection into a fluid conduit of the body, the apparatus comprising: a first pump device for supplying a first fluid to the body, a pulsing device for applying a pulse to the fluid to be injected, and a conduit for conveying the fluid incorporating the pulse into the fluid conduit of the body.

In one embodiment, the pulsing device may be a vibration source as discussed above, which may for example be connected to tubing that conveys fluid to the patient or connected to a syringe that powers the flow of fluid into the patient.

Preferably, the vibration source is arranged to propagate a vibration into the fluid to be injected, whilst reducing or eliminating vibration transmitted to the body via tubing conveying the fluid to the body. Hence, the vibration source may be isolated from the tubing and/or catheter by a damping device, A preferred damping device comprises a resilient and/or vibration absorbing material located between the vibration source and the tubing and/or catheter. In one possible arrangement, a vibration source is placed within tubing in communication with fluid flowing to the body, and a damped coupling is provided to support the vibration source whilst reducing or eliminating vibration transmitted to the body via the tubing. The damped coupling may take the form of a resilient and/or vibration absorbing material located about points of contact between the tubing and a support for the vibration source.

The frequency of vibration produced by the vibration source may be matched to the properties of the damping device or damped coupling, such that the damping reduces or eliminates vibrations at that particular frequency. The damped coupling and or damping device may comprise silicone rubber material, for example a silicone rubber foam.

In an alternative arrangement the pulsing device may be a second pump device for supplying a second fluid to the body, wherein the apparatus is arranged to supply the second fluid in a pulse such that the flow rate of the fluid injected into the body is temporarily increased.

As noted above, the temporarily increased flow rate in the injected fluid enables the injection to be monitored more easily. The apparatus may include a first reservoir for the first fluid. Where a second fluid is used, the apparatus may include a second reservoir for the second fluid, with the first and second pump devices being arranged to supply fluid from the respective reservoir. Preferably, the first pump device is arranged to supply the first fluid at a continuous flow rate.

In a preferred embodiment, the apparatus is for monitoring the intended injection of a fluid into a fluid conduit in the body. As discussed above in relation to the method of the invention, the addition of a pulse in the flow rate of the injected fluid enables effective monitoring of the injection. The apparatus may include connections from the first pump device and (if present) the second pump device to a catheter for supplying fluid into a blood vessel or other fluid conduit in the body.

Preferably, the apparatus comprises a monitoring device for monitoring a flow characteristic in the fluid conduit at a location downstream of the point of injection. The monitoring device may be a flow velocity measuring device for measuring the flow velocity in the fluid conduit at a location downstream of the point of injection. A measurement of the blood flow velocity enables the effect of the changed velocity of the injected fluid to be easily and non-invasively detected. By introducing a pulse to change the flow rate, the flow velocity measuring device can monitor the injection even where the first fluid flow rate is too low to produce a detectable change in the flow velocity in the fluid conduit when injected at a fixed rate. Alternatively or in addition, the monitoring device may be for monitoring turbulence, in the fluid flow in the flow conduit. The monitoring device may be an ultrasound monitoring device, and is preferably a Doppler ultrasound probe, as discussed above.

In other embodiments, the monitoring device may comprise a sound monitoring device for detecting the sound of the flow within the fluid conduit. For example, the apparatus may comprise a microphone and/or a stethoscope. Audible feedback can hence be provided to the operator, or a sound signal may be produced.

Preferably, the first pump device and/or the second pump device comprise(s) an automated injection device, such as an infusion pump. Hence, the reservoir may be a body of a syringe, and the pump device can comprise an actuatable plunger, for pumping fluid out of the syringe body. Where a vibration apparatus is used, this may be fixed to the plunger of the pump device for the first fluid, or it may be located on the tubing connecting the pump device to the patient. In one preferred embodiment, the first and second pump devices are provided in the form of an infusion pump apparatus for injecting multiple fluids, for example an infusion pump apparatus with two or more automated syringes on a rack.

The apparatus preferably includes a controller, such as a computer device, for receiving a signal from the monitoring device and providing feed back to the operator and/or feed back to the first and second pump devices (if present). The feed back can be a visual or audible indication of the measured flow characteristics, or an indication when there is a signal profile that results from possible problems with the injection. This may for example be an absence of an expected increase in flow velocity or reflection planes. The controller may be for handling feed back as in the method discussed above, and is preferably arranged to carry out such feed back processes.

Where a pulse of a second fluid is used, the second pump device is preferably arranged to introduce the pulse automatically, and is more preferably arranged to introduce a pulse at repeated intervals. The controller may also be for controlling the first and/or second pump devices. The controller is hence preferably programmed to operate the second pump device to repeat an injection of a pulse of fluid at a set flow rate and duration at a predetermined interval. The flow rate, duration and interval of the pulse may be as discussed above.

The first and second pump devices are preferably fluidly connected to a single catheter for conveying the first and second fluids to the fluid conduit.

The pump devices may be arranged such that the first fluid and the pulse of the second fluid are injected consecutively, with, for example, a period of injection of the first fluid, followed by a pulse of the second fluid, followed by recommencement of injection of the first fluid. Thus, during the course of injection of the first fluid, the second fluid is supplied in a pulse when the injection of the first fluid is paused. Preferably however, the pump devices are arranged such that the pulse of the second fluid is introduced whilst the injection of the first fluid proceeds continuously.

In a further aspect, the invention extends to the use of an apparatus as described above in relation to the second aspect. This may include a method comprising use of the apparatus for monitoring an ongoing injection of fluid.

The invention also encompasses computer program products containing instructions that when executed on a data processing apparatus will configure the data processing apparatus to carry out one of the methods discussed above. In a preferred embodiment this comprises software loadable onto or stored on a computer readable medium and consisting of computer readable program code for performing the method when the software is executed on a computer.

In preferred embodiments of the above aspects, an ultrasound probe having an array of two or more ultrasound sensors is used to monitor the flow characteristics in the fluid conduit. The array is arranged to be placed substantially transverse to the direction of flow of the fluid conduit so that at least one sensor of the array will be located over the conduit. With this arrangement, the accuracy with which the detector must be placed is reduced. It can be difficult to precisely locate a fluid conduit such as a blood vessel downstream of the point of infusion. This arrangement makes it simple to obtain a good signal Without time consuming repositioning of the probe. Provided that the ultrasound sensor is located approximately downstream of the infusion site, at least one sensor of the array will be located over the vessel, and so it is not necessary to precisely locate the blood vessel at the point of measurement prior to commencing the measurement. Instead, the flow characteristics can be detected by whichever sensor or sensors of the array are located over the blood vessel.

With this arrangement, it is preferable that the sensor of the array that is located over the blood vessel is automatically detected using the difference in signal between sensors. The signal from an ultrasound sensors varies depending on whether the sensor is located over the blood vessel or over ordinary tissue. The highest signal will be obtained at the sensor in the array that is receiving measurements of the moving fluid in the blood vessel. This sensor can thus be selected to be used to monitor the intended injection.

In a particularly preferred embodiment, the signal received by the sensor of the array that has the lowest signal strength is used as a baseline signal level. This signal level is subtracted from the signal level of the sensor that is used to monitor the flow velocity in the blood vessel. The use of a baseline signal level in this way aids in noise cancellation, and will help reduce or eliminate erroneous signals caused by movement, palpation or other sources of noise, including external sources such as electrical devices. The baseline sensor may alternatively be a separate sensor intended to be positioned away from the blood vessel for the purpose of detecting signal changes due to movement and noise.

In all the embodiments discussed above, the ultrasound probe may include a microphone, and/or the ultrasound signal may be used as the basis for a sound signal. This enables audible feed-back to be provided. Where a microphone is used, the operator can listen to distinguish different types of blood flow, for example arterial and venous flow. When the ultrasound signal is used, this enables audible feed-back representing the change in ultrasound signal strength to be given to the operator. Consequently, the operator can listen directly or indirectly for the sound of a change in the fluid flow characteristics when the pulse of second fluid is introduced. The operator can monitor the injection by ear, with any potential problem being audibly indicated, perhaps in addition to a visual indication.

The invention relates to monitoring an injection of fluid into a fluid conduit such as a blood vessel. However, in some circumstances the fluid will never enter the fluid conduit, and thus strictly speaking there is no injection of fluid. For example if the needle is badly located and directly injects into tissue instead of into the blood vessel. Thus, in the discussion above, reference is made to monitoring an intended injection, in order to encompass monitoring of both an injection that is successful in introducing fluid into the blood vessel, as well as an injection that for whatever reason is not successful.

The monitoring of an intended injection into a fluid conduit by measurement of turbulence in the flow in the fluid conduit is considered inventive in its own right, and therefore an additional broad aspect of the invention provides a method of monitoring an intended injection of a fluid into a fluid conduit comprising: monitoring turbulence in the fluid conduit at a location downstream of the point of injection. A further broad aspect of the invention provides an apparatus for monitoring an intended injection of a fluid into a fluid conduit comprising: a monitoring device for monitoring turbulence in the fluid conduit at a location downstream of the point of injection. When an injection is successful, the turbulence will increase. Preferably, a Doppler ultrasound technique is used to monitor turbulence, making use of the increase in reflection planes as described above. Feed back to an operator or an injection apparatus can be provided in a similar manner to that described above.

Preferred embodiments of the present invention will now be described by way of example only and with reference to the accompanying drawings in which:

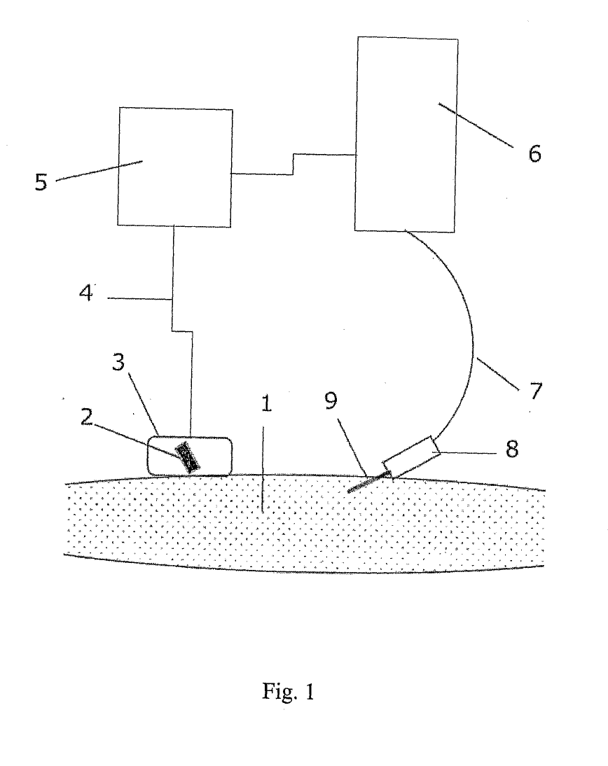

FIG. 1 shows schematically an arrangement for monitoring the injection of fluid in accordance with embodiments of the invention,

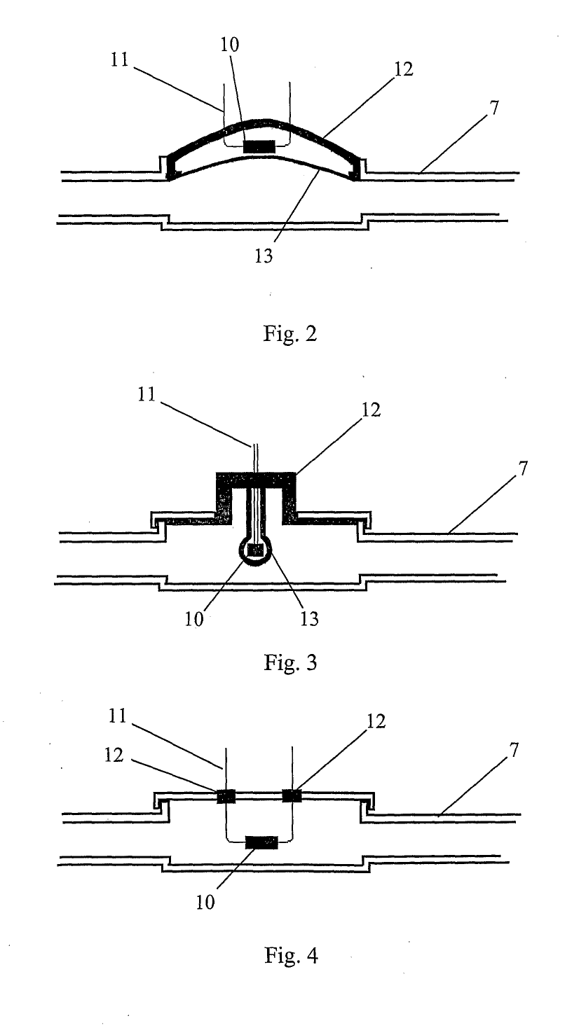

FIG. 2 is a first example arrangement of an apparatus for applying a vibration to the fluid to be injected,

FIG. 3 is a second example arrangement of an apparatus for applying a vibration to the fluid to be injected, and

FIG. 4 is a third example arrangement of an apparatus for applying a vibration to the fluid to be injected.

In FIG. 1, an injection monitoring apparatus is shown in use for monitoring the infusion of a fluid into a blood vessel in a patient's arm. A first fluid is infused into the patient's arm 1 from an infusion pump apparatus 6. The first fluid can be a cytotoxin. The arrangement of FIG. 1 can be used in relation to a first embodiment, which utilises a pulse of a second fluid introduced into the first fluid, or in relation to a second embodiment, which utilises a vibration applied to the first fluid. The main features and functionality of the injection apparatus is the same in each case.

In the first embodiment, the infusion pump apparatus 6 has at least two automatically controlled syringes, or a similar arrangement capable of supplying multiple fluid types to a patient. The infusion pump apparatus 6 hence also has a supply of a second fluid, typically saline, which can be infused into the patient's arm along with the first fluid. The first and second fluids are conveyed from the infusion pump apparatus 6 to a flexible tube 7, and then via the tube 7 to a cannula or venflon arrangement 8, which comprises a connector for connection to the flexible tube 7, and a fine bore tube 9 which has been inserted into a vein in a known manner.

The infusion pump apparatus 6 is controlled by an electronic controller 5, which varies the injection speed as required and starts and stops the injection of the first and the second fluids. The controller 5 thus initiates the injection of the first fluid, at the desired flow rate, when prompted by the operator as discussed below. The controller 5 also operates the pump for the second fluid to provide pulses at a preset rate and size. For example, pulses with a flow rate of 0.2 ml/s and a duration of 0.5 seconds are provided every 2 seconds throughout the course of the injection of the first fluid. Other pulse sizes and durations can be used as appropriate for the particular infusion pump characteristics.

An ultrasound Doppler probe 3 is placed above the same vein at a convenient distance downstream so as to be clear of the infusion site. The Doppler probe 3 consists of a number transducer elements 2 which in use are placed at an angle to the vein to create and detect a Doppler shift resulting from the flow of fluid in the vein.

The transducer elements 2 comprise ultrasound sensors arranged in an array, which is placed substantially transversely over the vein. The signal levels of the sensor elements in the array is measured, and this is used to select appropriate sensors to measure the flow characteristics in the vein and to provide a baseline signal for noise cancellation. The sensor in the array with the highest signal is selected to be used to as a first sensor, which measures the flow velocity. The sensor in the array that has the lowest signal strength can be used as a second sensor, for noise cancellation. As will be appreciated, the sensor with the lowest signal level will be a sensor that is not affected by the movement of fluid in the blood vessel. The signal level of the second sensor will be the result of background reflections from body tissue and noise from external sources and from body movement. This baseline signal can be deducted from the signal level of the first sensor in order to give a more accurate signal indicating the flow velocity in the blood vessel and changes in this flow velocity.

The probe 2 is connected via a flexible lead 4 to the controller 5 which includes a processor unit and display. This converts the output from the probe 3 into a form that may be displayed as an image on the display unit in a conventional manner. In addition it provides a digital signal proportional to the flow velocity detected by the probe 3. This value is then also displayed on the display.

The controller 5 receives data concerning the Doppler ultrasound measurements and injection parameters, and provides output signals to control the infusion pump apparatus 6. The Doppler ultrasound measurements can be processed to generate a flow velocity measurement and/or an indication of frequency response. This allows the controller 5 to detect the pulse of saline based on a velocity increase and/or an increase in reflection planes, depending on the particular injection conditions. It will be appreciated that various arrangements could be used to implement the required controller that monitors and controls the injection process. There can also be data passed back to the controller 5 from the infusion pump apparatus 6, such as data from a pressure sensor, which can be used to indicate if there is a potential problem with the infusion pump apparatus 6.

The operator inputs the desired injection flow rate and the duration of the injection into the processor unit of the controller 5. The size and duration of the pulses of the second fluid can be input by the operator, or alternatively they may be derived automatically based on the flow rate of the first fluid and/or the infusion pump specification. When the injection is to commence the operator inputs a start signal into the controller 5. This in turn transmits a start signal to the controller 5 which energises the infusion pump apparatus 6 and causes it to supply the first fluid at the desired flow rate, and to supply pulses of the second fluid. The processor unit receives an indication that a pulse of the second fluid has been supplied, or alternatively is arranged to expect such a pulse at a pre-programmed time compared to the start of the injection. The processing unit then monitors the signal produced by the Doppler ultrasound probe 3 as described above. If the measured flow velocity or reflection plane characteristics includes pulses corresponding to the pulses of the second fluid supplied by the infusion pump apparatus, then the injection is allowed to continue.

However, if an expected pulse is not received, or if the pulse is not of sufficient amplitude, then this indicates a problem with the injection. If the expected pulse or size of pulse in the signal from the Doppler ultrasound probe 3 is not detected, then the infusion will be stopped. This is achieved by sending a signal to the controller 5, which controls the infusion pump apparatus accordingly.

If the injection is proceeding satisfactorily then the controller 5 continues to monitor the signal from the Doppler ultrasound probe 3. If one of the expected pulses in the signal is not detected or is too small then the controller 5 can again stop or at least pause the injection by control of the infusion pump apparatus 6. Cytotoxins and chemotherapy drugs are typically injected in repeated doses, over a period of time. Thus, with this type of injected fluid it is invariably best to halt the injection if there is a suspected extravasation.

The processor unit includes signal processing means to classify the Doppler measurements and identify anomalies. This can be done using known techniques, such as neural networks as discussed by Guler I. and Übeyli E. in “A recurrent neural network classifier for Doppler ultrasound blood flow signals”, Pattern Recognition Letters, Volume 27, Issue 13, 1 Oct. 2006, Pages 15601571 or Support Vector Machines (SVMs) as discussed by Übeyli E in “Doppler ultrasound signals analysis using multiclass support vector machines with error correcting output codes”, Expert Syst. Appl. 33(3): 725733 (2007). Bayesian classifiers have also been used to classify medical Doppler signals, as discussed in “Bayesian Classifier for Medical Data from Doppler Unit” by Malek J., Acta Polytecnica, Vol. 46, no. 4/2006.

In a second embodiment, in place of a second fluid, a pulse is provided by the use of a vibration device such as a motor with an off-centre mass to produce oscillation or any other form of oscillation device. The vibration device is coupled with the fluid that is going to be injected. The vibration device can be attached to the plunger of the syringe that supplies the first fluid, although in preferred embodiments the vibration is applied by means of a vibration source 10 placed within the flexible tube 7, and passes into the fluid conduit in the body by propagation through the fluid as it is injected into the body. Example arrangements of vibration devices are described below in relation to FIGS. 2 to 4.

FIG. 2 shows a vibration source 10 placed adjacent to a part of the tubing 7 that conveys fluid from the injection pump 6 to the body. Wiring 11 connects the vibration source 10 to its power source (not shown) and to the controller 5, which controls activation of the vibration source 10 during the injection procedure. The vibration source 10 is encapsulated within a chamber that comprises a wall of resilient vibration reducing material 12 which encloses one side of the chamber and provides a damped coupling between the vibration source 10 and the tubing 7. Damping is provided by the characteristics of the resilient vibration reducing material 12 and by flexure permitted by the domed shape of the wall. The other wall of the chamber is a membrane 13 for propagation of vibration from the vibration source 10 into the fluid to be injected.

An alternative arrangement is shown in FIG. 3. Here, the vibration source 10 is supported from a wall of resilient vibration reducing material 12 and is within a bulb of membrane 13 at the end of an elongate membrane chamber. The elongate shape allows the wiring 11 and membrane chamber that support the vibration source 10 to flex and provide some damping and reduction of vibration transmitted to the tubing 7. The wall of resilient vibration reducing material 12 also has a damping effect.

The membrane elements 13 of FIGS. 2 and 3 can be used to isolate the vibration source 10 from the fluid to be injected, Hence the membrane 13 could be impermeable and could be filled with a fluid or other composition arranged for transmission of vibrations.

FIG. 4 shows an arrangement that does not utilise a membrane. In the arrangement of FIG. 4 the vibration source 10 is supported within the fluid in the tubing 7 by the wiring 11. The wiring 11 passes through a wall of the tubing 7 where it is coupled to the wall via blocks of resilient vibration reducing material 12, which provide the required reduction in the transmission of vibration to the tubing 7.

The vibration device supplies a vibration at a frequency that is preferably selected from frequencies between 10 Hz and 200 Hz. Vibration is applied throughout the injection process. In this embodiment the ultrasound Doppler probe 3, which is placed and operated in the same manner as for the first embodiment, is used to detect a corresponding frequency in the blood downstream of the injection. The controller 5 operates as in the first embodiment, aside from the fact that it detects the vibration and determines whether or not there is a problem with the injection based on the presence and optionally the level of the vibration in the blood flow. The pump apparatus 6 can be controlled by the controller 5 as in the first embodiment.

In addition to the pulses of saline used as the second fluid during the injection of the first fluid in the first embodiment above, saline can also be used alone prior to or after the injection of the first fluid in relation to either embodiment. If injected before the first fluid the saline acts to open the vein, and prepare it for the injection. When injected after the first fluid the saline can be used to push the first fluid further along the blood vessel by applying pressure behind it. Saline can also be used in this way in conjunction with the second embodiment of the invention.

As will be appreciated, the apparatus of the preferred embodiment is inherently capable of producing a measurement of a blood flow velocity, or other fluid flow velocities in fluid conduits in the human or animal body. Therefore, the processing unit 4 may usefully be provided with the ability to provide a straightforward velocity measurement, in addition to having the capability to detect extravasation and the like as discussed above.

The system shown in FIG. 1 is illustrated as using a wired connection 3 between the probe 2 and controller 5. In an alternative embodiment, data transmission between the probe 2 and controller 5 is by a wireless connection. The use of wireless data transmission is useful as it avoids a potential entanglement risk, and gives more freedom of movement by the patient and around the patient. Further, the various processing units and controllers of the embodiments described and claimed can be a single unit, such as a CPU, or could for convenience be separated. For example, local processing of ultrasound sensor signals could occur at the probe, enabling the first and second sensors to be selected and the baseline signal used to adjust for noise without the need to transmit data to and from the processing unit or controller 5 that controls the infusion pump apparatus 6.

Another modification to the system of FIG. 1 involves the use of sound. As is well known, venous and arterial flows have distinguishable sounds. A microphone can be provided with or as part of the ultrasound probe in order to enable the sound of blood vessels at the location of the probe to be heard. These sounds can be provided to the operator by head phones or by a speaker system. This allows the operator to more easily position the probe over the desired blood vessel. The sound of the blood flow can also be used to detect the expected pulses, i.e. as an alternative to or to double-check the results of the ultrasound measurement. In addition, the variation in signal characteristics from the ultrasound probe during the course of a procedure can be relayed as sound to the operator, i.e. the ultrasound signal can be used as the basis for a sound signal. This allows the pulses/vibrations in signal strength resulting from correct operation to be presented as audible feed-back to the operator. This audible feed-back can be used in addition to a visual signal and/or a separate alarm.

It will be appreciated that an apparatus could be provided with the ability to apply a vibration to the fluid to be injected, and with the ability to use a pulse of a second fluid. This would allow the operator to select the most effective mechanism for increasing the accuracy and reliability of the injection monitoring process for a particular injection regime or patient. It is also possible to combine the use of a pulse of a second fluid with a pulse in the form of a vibration in order to provide two redundant mechanisms for monitoring the injection.

We claim:

1. A method of monitoring the intended injection of a fluid into a fluid conduit in the body, wherein the fluid to be injected comprises a first fluid, the method comprising: introducing a pulse into the fluid to be injected and monitoring the flow characteristics of fluid in the fluid conduit in the body at a location downstream of the point of injection.

2. A method as claimed in claim 1, wherein the flow characteristics are monitored by using a Doppler ultrasound technique.

3. A method as claimed in claim 1, wherein the pulse is introduced by the addition of a pulse of a second fluid into the first fluid.

4. A method as claimed in claim 3, wherein the flow rate of the pulse of the second fluid is at least 0.1 ml/s and the pulse has a duration of at least 0.1 seconds.

5. A method as claimed in claim 3, comprising introducing the pulse automatically, for example by using an infusion pump apparatus or the like.

6. A method as claimed in claim 1, wherein the pulse comprises a vibration that is applied to the fluid to be injected.

7. A method as claimed in claim 6, comprising propagating a vibration through the fluid to be injected by using a vibration source coupled to the fluid.

8. A method as claimed in claim 7, comprising reducing or eliminating vibrations passed from the vibration source to tubing and/or a catheter conveying fluid to the body.

9. A method as claimed in claim 8, wherein the vibration source is isolated from the tubing and/or catheter by a resilient and/or vibration absorbing material.

10. A method as claimed in claim 1, comprising providing an alarm if an expected change in the fluid flow velocity in the flow conduit is not detected or if the amplitude of the change differs from an expected amplitude.

11. An apparatus for supplying fluid for injection into the body, the apparatus comprising: a first pump device for supplying a first fluid to the body, a pulsing device for applying a pulse to the fluid to be injected, and a conduit for conveying the fluid incorporating the pulse into the fluid conduit of the body.

12. An apparatus as claimed in claim 11, wherein the pulsing device comprises a second pump device for supplying a second fluid to the body, the apparatus comprising a first reservoir for the first fluid and a second reservoir for the second fluid, with the first and second pump devices being arranged to supply fluid from the respective reservoir wherein the apparatus is arranged to supply the second fluid in a pulse such that the flow rate of the fluid injected into the body is temporarily increased.

13. An apparatus as claimed in claim 11, wherein the pulsing device is a vibration source for introducing a vibration into the fluid to be injected.

14. An apparatus as claimed in claim 13 wherein the vibration source is arranged to propagate a vibration into the fluid to be injected whilst reducing or eliminating vibration transmitted to the body via tubing conveying the fluid to the body.

15. An apparatus as claimed in claim 14, wherein the vibration source is isolated from the tubing and/or catheter by a damping device.

16. An apparatus as claimed in claim 15, wherein the damping device comprises a resilient and/or vibration absorbing material located between the vibration source and the tubing and/or catheter.

17. An apparatus as claimed in claim 11, comprising a monitoring device for monitoring a flow characteristic in the fluid conduit at a location downstream of the point of injection.

18. An apparatus as claimed in claim 17, wherein the monitoring device is a Doppler ultrasound probe.

19. The use of an apparatus as claimed in claim 11 for monitoring an ongoing injection of fluid.

20. A method of supplying a fluid for injection into a fluid conduit in the body, wherein the fluid to be injected comprises a first fluid, the method comprising: introducing a pulse into the fluid to be injected in order to temporarily change the flow rate of the injected fluid and conveying the fluid the fluid incorporating the pulse into the fluid conduit of the body.

21. (canceled)

22. (canceled)
